(19) United States
(12) Patent Application Publication
(10) Pub. No.: US 2023/0389575 A1
(43) Pub. Date: Dec. 7, 2023

(54) Pet Food Compositions

(71) Applicant: Hill's Pet Nutrition, Inc., Topeka, KS (US)

(72) Inventors: Dennis JEWELL, Lawrence, KS (US); Matthew JACKSON, Topeka, KS (US)

(73) Assignee: Hill's Pet Nutrition, Inc., Topeka, KS (US)

(21) Appl. No.: 18/268,722

(22) PCT Filed: Dec. 19, 2021
(86) PCT No.: PCT/US2021/064254

§ 371 Date: Jun. 21, 2023

Related U.S. Application Data
(60) Provisional application No. 63/129,325, filed on Dec. 22, 2020.

(51) Int. Cl. A23K 20/158; A23K 20/163; A23K 10/30; A23K 50/40; A61K 31/716; A61K 31/23; A61K 31/197; A61K 36/906; A61K 36/185; A61K 36/21; A61K 31/202

(52) U.S. Cl. CPC A23K 20/158; A23K 20/163; A23K 10/30; A23K 50/40; A61K 31/716; A61K 31/23; A61K 31/197; A61K 36/906; A61K 36/185; A61K 36/21; A61K 31/202

(57) ABSTRACT

Described herein are pet food compositions and methods for using them. Such compositions may comprise beta glucans, one or more medium chain saturated fatty acids and one or more omega 3 polyunsaturated fatty acids, and betaine. The pet food composition may further comprise botanicals. The methods include feeding the pet an effective amount of the pet food composition.

### CROSS-REFERENCE TO RELATED APPLICATION

This application claims the benefit of priority from U.S. Provisional Application No. 63/129,325, filed Dec. 22, 2020, the contents of which are hereby incorporated herein by reference in their entirety.

### BACKGROUND

The well-being of domestic animals is closely related to their feeding. Correct feeding should result in a fit and healthy pet. To achieve correct feeding, one may utilize certain ingredients and concentrations of those ingredients which yield beneficial effects for the animal.

Such beneficial effects may include weight loss, fat loss, retention of lean mass, and/or high improvement in the lean mass to body weight ratio of the animal.

It would therefore be desirable to provide a pet food composition which may beneficially affect animals suffering from being overweight.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Applicants have discovered that utilization of certain ingredients within a pet food composition provides for effective health benefits. In one aspect, the health benefit may be to decrease the weight of the animal. In another aspect, the health benefit may be to decrease the fat of the animal. In another aspect, the health benefit may be to improve the lean mass to fat mass ratio, such as increasing the lean mass to fat ratio, of the animal.

Thus, in one aspect, the invention is a pet food composition comprising 1-3, 1-6 beta glucan and 1-3, 1-4 beta glucan present in a total amount equal to at least about 0.8%, based on the weight of the pet food composition, wherein the 1-3, 1-4 beta glucan is at least about 83%, by weight, of the two beta glucans; two or more medium chain saturated fatty acids and two or more omega-3 polyunsaturated fatty acids present in a total amount of at least about 0.5%, based on the weight of the pet food composition, wherein the two or more medium chain saturated fatty acids are at least 36%, by weight, of these fatty acids; and betaine present in an amount of at least about 0.5%, based on the weight of the pet food composition. In certain embodiments, the 1-3, 1-6 beta glucan and 1-3, 1-4 beta glucan are present in an amount from about 0.8% to about 5%, based on the weight of the pet food composition. In certain embodiments, the 1-3, 1-6 beta glucan and 1-3, 1-4 beta glucan are present in an amount from about 0.8% to about 2%, based on the weight of the pet food composition. In certain embodiments, the 1-3, 1-4 beta glucan is present in an amount from 83% to 95%, by weight, of the two beta glucans. In certain embodiments, the 1-3, 1-4 beta glucan is present in an amount of about 94%, by weight, of the two beta glucans. In certain embodiments, the two or more medium chain saturated fatty acids comprise a C8 fatty acid and a C10 fatty acid. In certain embodiments, the two or more omega 3 polyunsaturated fatty acids comprise eicosapentaenoic acid (EPA) and docosahexaenoic acid (DHA). In certain embodiments, the two or more medium chain saturated fatty acids and the two or more omega 3 polyunsaturated fatty acids are present in a total amount of from about 0.5% to 7%, based on the weight of the pet food composition. In certain embodiments, the two or more medium chain saturated fatty acids and the two or more omega 3 polyunsaturated fatty acids are present in a total amount of about based on the weight of the pet food composition. In certain embodiments, the C8 and C10 comprise from about 36% to about 60%, by weight, of the two or more medium chain saturated fatty acids and the two or more omega 3 polyunsaturated fatty acids. In certain embodiments, the C8 and C10 comprise from about 36% to about 45%, by weight, of the two or more medium chain saturated fatty acids and the two or more omega 3 polyunsaturated fatty acids. In certain embodiments, the C8 and C10 comprise about 36%, by weight, of the two or more medium chain saturated fatty acids and the two or more omega 3 polyunsaturated fatty acids. In certain embodiments, the betaine is present in an amount of about 0.5% to about 2%, based on the weight of the pet food composition. In certain embodiments, the betaine is present in an amount of about based on the weight of the pet food composition.

In certain embodiments, the pet food composition further comprises lignin present in an amount of at least 1.25%, based on the weight of the pet food composition. In certain embodiments, the lignin is present in an amount of about 1.25% to 5%, based on the weight of the pet food composition. In certain embodiments, the lignin is present in an amount of about 1.25%, based on the weight of the pet food composition.

In certain embodiments, the pet food composition further comprises botanicals. In certain embodiments, the botanicals comprise cranberry pomace, ginger root, pomegranate and green tea. In certain embodiments, the cranberry pomace, ginger root, pomegranate and green tea are present in a total amount of about 1.2% or more, based on the weight of the pet food composition. In certain embodiments, the composition further comprises beet pulp. In certain embodiments, the beet pulp is present in an amount of about 1% or more, based on the weight of the pet food composition. In certain embodiments, the beet pulp is present in an amount of about 1%, based on the weight of the pet food composition.

In further embodiments, the invention is directed to a method for increasing the lean to fat ratio in a companion animal, comprising feeding an effective amount of a pet food composition comprising 1-3, 1-6 beta glucan and 1-3, 1-4 beta glucan present in an amount equal to at least about 0.8%, based on the weight of the pet food composition, wherein the 1-3, 1-4 beta glucan is at least about 83%, by weight, of the two beta glucans; two or more medium chain saturated fatty acids and two or more omega 3 polyunsaturated fatty acids are present in a total amount of at least about 0.5%, based on the weight of the pet food composition, wherein the two or more medium chain saturated fatty acids are present in an amount of at least 36%, by weight, of these fatty acids; and betaine is present in an amount of at least about 0.5%, based on the weight of the pet food composition. In certain embodiments, the 1-3, 1-6 beta glucan and 1-3, 1-4 beta glucan are present in an amount from about 0.8% to about 5%, based on the weight of the pet food composition. In certain embodiments, the 1-3, 1-6 beta glucan and 1-3, 1-4 beta glucan are present in an amount from about 0.8% to about 2%, based on the weight of the pet food composition. In certain embodiments, the 1-3, 1-4 beta glucan is from 83% to 95%, by weight, of the two beta glucans. In certain embodiments, the 1-3, 1-4 beta glucan is about 94%, by weight, of the two beta glucans. In certain embodiments, the two or more medium chain saturated fatty acids comprise a C8 fatty acid and a C10 fatty acid. In certain embodiments, the two or more omega 3 polyunsaturated fatty acids comprise eicosapentaenoic acid (EPA) and docosahexaenoic acid (DHA). In certain embodiments, the two or more medium chain saturated fatty acids and the two or more omega 3 polyunsaturated fatty acids are present in a total amount of from about 0.5% to 7%, based on the weight of the pet food composition. In certain embodiments, the two or more medium chain saturated fatty acids and the two or more omega 3 polyunsaturated fatty acids are present in a total amount of about 0.5%, based on the weight of the pet food composition. In certain embodiments, the C8 and C10 comprise from about 36% to about 60%, by weight, of the two or more medium chain saturated fatty acids and the two or more omega 3 polyunsaturated fatty acids. In certain embodiments, the C8 and C10 comprise from about 36% to about 45%, by weight, of the two or more medium chain saturated fatty acids and the two or more omega 3 polyunsaturated fatty acids. In certain embodiments, the C8 and C10 comprise about 36%, by weight, of the two or more medium chain saturated fatty acids and the two or more omega 3 polyunsaturated fatty acids. In certain embodiments, the betaine is present in an amount of about 0.5% to about 2%, based on the weight of the pet food composition. In certain embodiments, the betaine is present in an amount of about 0.5%, based on the weight of the pet food composition.

In further embodiments, the method utilizes a pet food composition further comprising lignin present in an amount of at least 1.25%, based on the weight of the pet food composition. In certain embodiments, the lignin is present in an amount of about 1.25% to 5%, based on the weight of the pet food composition. In certain embodiments, the lignin is present in an amount of about 1.25%, based on the weight of the pet food composition. In certain embodiments, the pet food composition further comprises botanicals. In certain embodiments, the botanicals comprise cranberry pomace, ginger root, pomegranate and green tea. In certain embodiments, the cranberry pomace, ginger root, pomegranate and green tea are present in a total amount of about 1.2% or more, based on the weight of the pet food composition. In certain embodiments, the pet food composition further comprises beet pulp. In certain embodiments, the beet pulp is present in an amount of about 1% or more, based on the weight of the pet food composition. In certain embodiments, the beet pulp is present in an amount of about 1%, based on the weight of the pet food composition.

In certain embodiments, the companion animal is a dog. In certain embodiments, the companion animal is a cat.

Further areas of applicability of the present invention will become apparent from the detailed description provided hereinafter. It should be understood that the detailed description and specific examples, while indicating the typical embodiments of the invention, are intended for purposes of illustration only and are not intended to limit the scope of the invention.

### BRIEF DESCRIPTION OF THE DRAWINGS

The detailed description of the invention will be better understood when read in conjunction with the appended drawings. It should be understood, however, that the invention is not limited to the precise arrangements and instrumentalities of the embodiments shown in the drawings.

FIG. 1 depicts a chart showing the modelled effects on weight and inflammation during the weight maintenance and weight loss time periods;

FIG. 2 depicts both a chart showing the change in mass during the weight maintenance and weight loss time periods and a table expressing the quantified results;

FIG. 3 depicts both a chart showing the change in fat during the weight maintenance and weight loss time periods and a table expressing the quantified results;

FIG. 4 depicts charts showing the change in lean mass during the weight maintenance and weight loss time periods;

FIG. 5 depicts a chart showing the change in the lean to fat ratio during weight maintenance and weight loss time periods;

FIGS. 6A-6C depict the effects of the test food composition on pro-inflammatory lipid biomarkers; and

FIG. 7 depicts charts showing effects on the test food on inflammatory cytokines IL-13, Fas-L and IL-2.

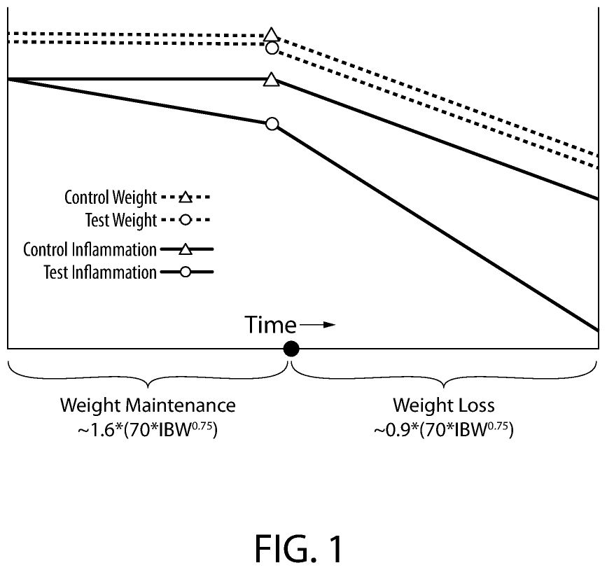

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other applications and methods. It is to be understood that the invention is not limited in its application to the details of any particular embodiment shown. The terminology used herein is for the purpose of description and not to limit the invention, its application, or uses.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, “containing”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range.

Unless otherwise specified, all percentages and amounts expressed herein and elsewhere in the specification should be understood to refer to percentages by weight of the total composition. Reference to a molecule, or to molecules, being present at a “wt. %” refers to the amount of that molecule, or molecules, present in the composition based on the total weight of the composition.

According to the present application, use of the term “about” in conjunction with a numeral value refers to a value that may be +/−5% of that numeral. As used herein, the term “substantially free” is intended to mean an amount less than about 5.0 weight %, less than 3.0 weight %, 1.0 wt. %; preferably less than about 0.5 wt. %, and more preferably less than about 0.25 wt. % of the composition.

As used herein, the term “effective amount” refers to an amount that is effective to elicit the desired biological response, including the amount of a composition that, when administered to a subject, is sufficient to achieve an effect toward the desired result. The effective amount may vary depending on the composition, the disease, and its severity and the age, weight, etc., of the subject to be treated. The effective amount can include a range of amounts. As is understood in the art, an effective amount may be in one or more doses, i.e., a single dose or multiple doses may be required to achieve the desired endpoint.

Unless defined otherwise, all technical and scientific terms used herein have the same meanings as commonly understood by one of ordinary skill in the art to which this invention belongs. All patents, patent applications, publications, and other references cited or referred to herein are incorporated by reference in their entireties for all purposes. In the event of a conflict in a definition in the present disclosure and that of a cited reference, the present disclosure controls.

The present disclosure is directed toward pet food compositions and methods of using such pet food compositions for the treatment of domestic pets. In certain embodiments, the pet is a dog. In other embodiments, the dog suffers from being overweight. In other embodiments, the dog is obese. A dog having a fat percentage of greater than 30% using a dual-energy absorptiometry (DEXA) scan may in some cases be identified as obese.

The present inventors have surprisingly and unexpectedly discovered that providing animals a pet food diet comprising beta glucans, preferably beta glucans having beta 1.3 links (e.g., 1-3, 1-6 beta glucan and 1-3, 1-4 beta glucan), present in an amount equal to or above about 0.8 wt. %, based on the weight of the pet food composition; and two or more medium chain saturated fatty acids and two or more omega 3 polyunsaturated fatty acids present in a total amount equal to or above about 0.5 wt. % or more, based on the weight of the pet food composition, provides for enhanced health benefit for the animal. It was further discovered that certain proportions and ratios of the beta glucans, medium chain saturated fatty acids, and omega-3 fatty acids may further enhance certain the benefits for pets. For example, the pet food composition may be formulated such that 1-3, 1-4 beta glucan is at least 83%, by weight, of the total amount of the two beta glucans; the two or more medium chain saturated fatty acids are at least 36%, by weight, of these fatty acids; and/or the pet food composition comprises betaine present in an amount of about 0.5% or more, based on the weight of the pet food composition. Such enhanced health benefit may be exemplified by numerous aspects. In a first aspect, the health benefit may be to decrease the weight of the animal. In another aspect, the health benefit may be to decrease the fat of the animal. In another aspect, the health benefit may be to retain the lean mass of the animal. In another aspect, the health benefit may be to improve the lean mass to fat ratio of the animal.

In one aspect, the present disclosure provides pet food compositions include beta glucans. Preferably the beta glucans comprise beta 1.3 links (e.g., 1-3, 1-6 beta glucan and 1-3, 1-4 beta glucan). For instance, in one embodiment, the pet food composition may comprise 1-3, 1-6 beta glucan and 1-3, 1-4 beta glucan present in an amount equal to or above about 0.8 wt. %, based on the weight of the pet food composition, wherein the 1-3, 1-4 beta glucan is at least 83%, by weight, of the two beta glucans; C8 fatty acid, C10 fatty acid, EPA and DHA present in a total amount equal to or above about 0.5 wt. % or more, based on the weight of the pet food composition, wherein C8 and C10 are at least 36 wt. %, by weight, of these fatty acids; and betaine present in an amount of about 0.5% or more, based on the weight of the pet food composition.

The one or more beta glucans (e.g., 1-3, 1-6 beta glucan and 1-3, 1-4 beta glucan) may be present at various amounts or concentrations. In one embodiment, the one or more beta glucans (such as, 1-3, 1-6 beta glucan and 1-3, 1-4 beta glucan) may be present in an amount of from about 0.8% to about 5%, based on the weight of the pet food composition. For example, the one or more beta glucans (e.g., 1-3, 1-6 beta glucan and 1-3, 1-4 beta glucan) may be present in an amount of about 0.8 weight %, about 1.0 weight %, about 1.2 weight %, about 1.4 weight %, about 1.6 weight %, about 1.8 weight %, about 2.0 weight %, about 2.2 weight %, about 2.4 weight %, about 2.6 weight %, about 2.8 weight %, about 3.0 weight %, about 3.2 weight %, about 3.4 weight %, about 3.6 weight %, about 3.8 weight %, about 4.0 weight %, about 4.2 weight %, about 4.4 weight %, about 4.6 weight %, about 4.8 weight %, about 5.0 weight %, or any range formed therefrom. In another example, the one or more beta glucans, such as 1-3, 1-6 beta glucan and 1-3, 1-4 beta glucan, may be present in an amount of from about 0.5 wt. % to about 6 wt. %, about 0.5 wt. % to about 5.0 wt. %, about 0.8 wt. % to about 5.0 wt. %, or about 0.8 wt. % to about 2.0 wt. %, based on the weight of the pet food composition. In further embodiments, the one or more glucans (e.g., 1-3, 1-6 beta glucan and 1-3, 1-4 beta glucan) are present in an amount of about 0. wt. 5% or more, about 0.8 wt. % or more, about 1.0 wt. % or more, or about 0.5 wt. % or more up to about 6.0 wt. %, based on the weight of the pet food composition. In further embodiments, the one or more glucans (such as 1-3, 1-6 beta glucan and 1-3, 1-4 beta glucan) are present in an amount of about 0.5 wt. % to 5 wt. %, about 0.8 wt. % to about 5 wt. %, about 0.8 wt. % to about 4 wt. %, or about 0.8 wt. % to about 3 wt. %, based on the weight of the pet food composition.

The amount or concentration of 1-3, 1-4 beta glucan with respect to total 1-3, 1-6 beta glucan and 1-3, 1-4 beta glucan amount or concentration may vary. In one embodiment, 1-3, 1-4 beta glucan may be present in an amount of from about 83 wt. % to about 95 wt. %, by weight, of the two beta glucans. For example, 1-3, 1-4 beta glucan may be present in an amount of about 83 wt. %, about 85 wt. %, about 87 wt. %, about 90 wt. %, about 92 wt. %, about 93 wt. %, about 94 wt. %, about 95 wt. %, or any range formed therefrom, by weight of the two glucans. In other embodiments, 1-3, 1-4 beta glucan may be present in an amount of from 83 wt. % to about 95 wt. %, or about 83 wt. % to about 92 wt. %, or about 83 wt. % to about 90 wt. %, or about 83 wt. % to about 88 wt. %, or about 83 wt. % to about 85 wt. %, by weight, of the two glucans. In certain embodiments, the 1-3, 1-4 beta glucan is present at about 94%, by weight, of the two beta glucans.

The medium chain saturated fatty acids typically comprise a glycerol molecule ester-linked to three saturated fatty acid groups, preferably with each fatty acid group having a carbon chain of 6 to 12 carbons. One or more of the saturated fatty acid groups may comprise a carbon chain of 6 to 11 carbons, 6 to 10 carbons, 6 to 9 carbons, 6 to 8 carbons; 7 to 12 carbons, 7 to 11 carbons, 7 to 10 carbons, 7 to 9 carbons, 7 or 8 carbons; 8 to 12 carbons, 8 to 11 carbons, 8 to 10 carbons, or 8 or 9 carbons. Although the three fatty acid groups of the medium chain saturated fatty acid may be the same, in some embodiments one or more of the fatty acid groups are different from another fatty acid group. The medium chain saturated fatty acids used in the pet food compositions may be obtained from various sources. The pet food composition may include one or more medium fatty acid selected from caproic (hexanoic acid), caprylic (octanoic acid), capric (decanoic acid), lauric (dodecanoic acid), or a combination of two or more thereof. In some cases, two or more medium fatty acids selected from caproic (hexanoic acid), caprylic (octanoic acid), capric (decanoic acid), lauric (dodecanoic acid), or a combination of two or more thereof are present in the pet food composition.

Omega-3 polyunsaturated fatty acids typically comprise a member of a group of polyunsaturated fatty carboxylic acids. In general, the omega-3 fatty acids contain 12-26 carbon atoms with methylene-interrupted double bonds, one of which is between the 3^{rd} and 4^{th} carbon atoms as counted from the methyl end of the fatty acid molecule. The omega-3 fatty acids may have an aliphatic tail of 16 to 24 carbons, 17 to 24 carbons, 18 to 24 carbons, 19 to 24 carbons, 20 to 24 carbons; 16 to 23 carbons, 17 to 23 carbons, 18 to 23 carbons, 19 to 23 carbons, 20 to 23 carbons; 16 to 22 carbons, 17 to 22 carbons, 18 to 22 carbons, 19 to 22 carbons, or 20 to 22 carbons. Preferably, the pet food composition comprises an omega-3 fatty acid having at least one aliphatic tail of 18 to 22 carbons. The omega-3 fatty acid may comprise linolenic acid, stearidonic acid, eicosatetraenoic acid, eicosapentaenoic acid, docosapentaenoic acid, docosahexaenoic acid, or a combination of two or more thereof. In at some examples, the pet food composition comprises omega-3 fatty acids selected from EPA, DHA, alpha-linolenic acid (ALA), and derivatives thereof. In certain embodiments, omega-3 fatty acids may be included in pet food compositions as components of triglycerides. Additional non-limiting examples of derivatives include salts and esters, such as branched or unbranched and/or saturated or unsaturated C1-C30 alkyl and cycloalkyl esters of omega-3 fatty acids.

The pet food compositions include two or more medium chain saturated fatty acids and two or more omega 3 polyunsaturated fatty acids. In certain embodiments, a C8 fatty acid, a C10 fatty acid, EPA and DHA are included. Fatty acids with 8 carbon atoms may be referred to herein as “C8 fatty acid” or “C8.” Fatty acids with 10 carbon atoms may be referred to herein as “C10 fatty acid” or “C10.”

A non-limiting example of a saturated C8 fatty acid is caprylic acid. The pet food composition may comprise one or more C8 fatty acids. The C8 fatty acid may be present at various amounts or concentrations. In one embodiment, C8 may be present in an amount of from about wt. % to about 6.5 wt. %, based on the weight of the pet food composition. For example, C8 may be present in an amount of about 0.5 weight %, about 1.0 weight %, about 1.5 weight %, about 2.0 weight %, about 2.5 weight %, about 3.0 weight %, about 3.5 weight %, about 4.0 weight %, about 4.5 weight %, about 5.0 weight %, about 5.5 weight %, about 6.0 weight %, about 6.5 weight % or any range formed therefrom, based on the total weight of the pet food composition. In another example, C8 may be present in an amount of from about 0.5 wt. % to about 6 wt. %, about 0.8 wt. % to about 5.5 wt. %, about 1.0 wt. % to about 5.0 wt. %, or about 1.5 wt. % to about 4 wt. %, based on the weight of the pet food composition. In further embodiments, C8 is present in an amount of about 0.5 wt. % or more, about 1.5 wt. % or more, about 2.5 wt. % or more, or about 2.7 wt. % or more up to about 6.3 wt. %, based on the weight of the pet food composition. In further embodiments, C8 is present in an amount of about 0.5 wt. % to 6 wt. %, about 1 wt. % to about 5 wt. %, about 1 wt. % to about 4 wt. %, about 1 wt. % to about 3 wt. %, or about 1 wt. % to about 2 wt. %, based on the weight of the pet food composition.

A non-limiting example of a C10 fatty acid is capric acid. In certain embodiments, the pet food composition may comprise one or more C10 fatty acids. The C10 fatty acid may be present at various amounts or concentrations. In one embodiment, C10 may be present in an amount of from about 0.5 wt. % to about 6.5 wt. %, based on the weight of the pet food composition. For example, C10 may be present in an amount of about 0.5 weight %, about 1.0 weight %, about 1.5 weight %, about 2.0 weight %, about 2.5 weight %, about 3.0 weight %, about 3.5 weight %, about 4.0 weight %, about 4.5 weight %, about 5.0 weight %, about 5.5 weight %, about 6.0 weight %, about 6.5 weight %, or any range formed therefrom, based on the total weight of the pet food composition. In another example, C10 may be present in an amount of from about 0.5 wt. % to about 6 wt. %, about 0.8 wt. % to about 5.0 wt. %, about 1.0 wt. % to about 4.0 wt. %, or about 1.0 wt. % to about 3 wt. %, based on the weight of the pet food composition. In further embodiments, C10 is present in an amount of about 0.5 wt. % or more, about 1.5 wt. % or more, about 2.5 wt. % or more, or about 2.7 wt. % or more up to about 6.3 wt. %, based on the weight of the pet food composition. In further embodiments, C10 is present in an amount of 0.5 wt. % to 6 wt. %, about 1 wt. % to about 5 wt. %, about 1 wt. % to about 4 wt. %, about 1 wt. % to about 3 wt. %, or about 1 wt. % to about 2 wt. %, based on the weight of the pet food composition.

The pet food compositions may comprise one or more omega-3 fatty acids, preferably two or more omega-3 fatty acids, typically in an amount of about 0.5% or more, based on the weight of the pet food composition. For example, omega-3 fatty acid may be present in the pet food compounds in an amount of from about 0.5 wt. % to about 10 wt. %, about 0.5 wt. % to about 9 wt. %, about 0.5 wt. % to about 8 wt. %, about 0.5 wt. % to about 7 wt. %, about 0.5 wt. % to about 6 wt. %, about 0.5 wt. % to about 5 wt. %, about 0.5 wt. % to about 4 wt. %, about 0.5 wt. % to about 3 wt. %, about 0.5 wt. % to about 2 wt. %; about 1 wt. % to about 10 wt. %, about 1 wt. % to about 9 wt. %, about 1 wt. % to about 8 wt. %, about 1 wt. % to about 7 wt. %, about 1 wt. % to about 6 wt. %, about 1 wt. % to about 5 wt. %, about 1 wt. % to about 4 wt. %, about 1 wt. % to about 3 wt. %, about 1 to about 2 wt. %; about 2 wt. % to about 10 wt. %, about 2 wt. % to about 9 wt. %, about 2 wt. % to about 8 wt. %, about 2 wt. % to about 7 wt. %, about 2 wt. % to about 6 wt. %, about 2 wt. % to about 5 wt. %, about 2 wt. % to about 4 wt. %, about 2% to about 3 wt. %; about 3 wt. % to about 10 wt. %, about 3 wt. % to about 9 wt. %, about 3 wt. % to about 8 wt. %, about 3 wt. % to about 7 wt. %, about 3 wt. % to about 6 wt. %, about 3 wt. % to about 5 wt. %, about 3 wt. % to about 4 wt. %; about 4 wt. % to about 10 wt. %, about 4 wt. % to about 9 wt. %, about 4 wt. % to about 8 wt. %, about 4 wt. % to about 7 wt. %, about 4 wt. % to about 6 wt. %, or about 4 wt. % to about 5 wt. %, based on the total weight of the pet food composition.

The omega-3 fatty acids may have an aliphatic tail of 16 to 24 carbons, 17 to 24 carbons, 18 to 24 carbons, 19 to 24 carbons, 20 to 24 carbons; 16 to 23 carbons, 17 to 23 carbons, 18 to 23 carbons, 19 to 23 carbons, 20 to 23 carbons; 16 to 22 carbons, 17 to 22 carbons, 18 to 22 carbons, 19 to 22 carbons, or 20 to 22 carbons. Preferably, the pet food composition comprises an omega-3 fatty acid having at least one aliphatic tail of 18 to 22 carbons. The omega-3 fatty acid may comprise linolenic acid, stearidonic acid, eicosatetraenoic acid, eicosapentaenoic acid, docosapentaenoic acid, docosahexaenoic acid, or a combination of two or more thereof.

The omega-3 fatty acid may comprise at least one omega-3 C18 fatty acid. An example of an omega-3 C18 fatty acid is alpha-linolenic acid (ALA). The pet food composition may comprise one or more omega-3 C18 fatty acids. The omega-3 C18 fatty acid may be selected from alpha-linolenic acid, stearidonic acid, and a combination thereof. The omega-3 C18 fatty acid may be present at various amounts or concentrations. Eicosapentaenoic acid (EPA) is an omega-3 fatty acid. It is found in the flesh of cold-water fish, including mackerel, herring, tuna, halibut, salmon, cod liver, whale blubber, or seal blubber. Docosahexaenoic acid (DHA) is an omega-3 fatty acid. It is found in the meat of cold-water fish, including mackerel, herring, tuna, halibut, salmon, cod liver, whale blubber, and seal blubber. It can also be produced by algae.

The two or more medium chain saturated fatty acids and two or more omega-3 polyunsaturated fatty acids may be present at various amounts or concentrations. In one embodiment, the two or more medium chain saturated fatty acids and two or more omega-3 polyunsaturated fatty acids may be present in a total amount of at least 0.5 wt. %, based on the weight of the pet food composition. For example, the two or more medium chain saturated fatty acids and two or more omega-3 polyunsaturated fatty acids may be present in an amount of about wt. %, about 0.7 wt. %, about 1.0 wt. %, about 1.2 wt. %, about 1.5 wt. %, about 1.8 wt. %, about 2 wt. %, about 2.3 wt. %, about 2.5 wt. %, about 2.8 wt. %, about 3.0 wt. %, or about 3.5 wt. %, based on the weight of the pet food composition. In other embodiments, the two or more medium chain saturated fatty acids and two or more omega-3 polyunsaturated fatty acids may be present in a total amount of from about 0.5 wt. % to about 8.0 wt. %, from about 0.5 wt. % to about 7.0 wt. %, from about 0.5 wt. % to about 5.0 wt. %, from about 0.5 wt. % to about 3.0 wt. %, or from about 0.5 wt. % to about 1.0 wt. %, based on the weight of the pet food composition.

The amount or concentration of C8 and C10 with respect to total C8 fatty acid, C10 fatty acid, EPA and DHA amount or concentration may vary. In one embodiment, C8 and C10 may be present in an amount of at least about 36%, by weight, of the total C8 fatty acid, C10 fatty acid, EPA and DHA amount or concentration. For example, C8 and C10 may be present in an amount of about 36%, about 38%, about 40%, about 42%, about 44%, about 46%, about 50%, or about 60%, by weight, of the total amount of C8 fatty acid, C10 fatty acid, EPA and DHA amount or concentration. In other embodiments, C8 and C10 may be present in an amount of from 36% to about 85%, about 36% to about 75%, about 36% to about 65%, about 36% to about 55%, or about 36% to about 45%, by weight, of the total amount of C8 fatty acid, C10 fatty acid, EPA and DHA amount or concentration. In other embodiments, C8 and C10 may be present in an amount of from 36% to about 60%, about 36% to about 70%, about 36% to about 60%, about 36% to about 50%, or about 36% to about 45%, by weight, of the total C8 fatty acid, C10 fatty acid, EPA and DHA amount or concentration. In other embodiments, C8 and C10 may be present in an amount of about 36%, by weight, of the total C8 fatty acid, C10 fatty acid, EPA and DHA amount or concentration.

The betaine may be present at various amounts or concentrations. In one embodiment, betaine may be present in an amount of at least about 0.5%, based on the weight of the pet food composition. For example, betaine may be present in an amount of about 0.5 weight %, about 0.7 weight %, about 1.0 weight %, about 1.2 weight %, about 1.4 weight %, about 1.6 weight %, about 1.8 weight %, about 2.0 weight %, about 2.2 weight %, about 2.4 weight %, about 2.6 weight %, about 3.0 weight %, about 3.5 weight %, about 4.0 weight %, about 4.5 weight %, about 5.0 weight %, or any range formed therefrom, based on the total weight of the pet food composition. In another example, betaine may be present in an amount of from about 0.5 wt. % to about 7.0 wt. %, about 0.5 wt. % to about 6.5 wt. %, about 0.5 wt. % to about 6.0 wt. %, about 0.5 wt. % to about 5.0 wt. %, about 0.5 wt. % to about 4.0 wt. %, or about 0.5 wt. % to about 3.0 wt. %, based on the weight of the pet food composition. In further embodiments, betaine is present in an amount of about 0.3 wt. % or more, about 0.5 wt. % or more, about 0.8 wt. % or more, or about 1 wt. % or more up to about 5 wt. %, based on the weight of the pet food composition. In further embodiments, betaine is present in an amount of about 0.5 wt. % to 4 wt. %, about 0.5 wt. % to about 3 wt. %, about 0. wt. 5% to about 2 wt. %, or about 0.5 wt. % to about 1 wt. %, based on the weight of the pet food composition. In certain embodiments, betaine is present in an amount of about 0.5 wt. %, based on the weight of the pet food composition.

The pet food compositions may include lignin. The lignin may be present at various amounts or concentrations. In one embodiment, lignin may be present in an amount of at least about 1.25 wt. %, based on the weight of the pet food composition. For example, lignin may be present in an amount of about 1.25 weight %, about 1.5 weight %, about 1.8 weight %, about 2.0 weight %, about 2.5 weight %, about 3.0 weight %, about 3.5 weight %, about 4.0 weight %, about 4.5 weight %, about 5.0 weight %, about 5.5 weight %, about 6.0 weight %, about 6.3 weight %, or a range formed therefrom, based on the total weight of the pet food composition. In another example, lignin may be present in an amount of from about 1.25 wt. % to about 6 wt. %, about 1.25 wt. % to about 5.5 wt. %, about 1.25 wt. % to about 5.0 wt. %, or about 1.25 wt. % to about 4 wt. %, based on the weight of the pet food composition. In further embodiments, lignin is present in an amount of about 1.25 wt. % or more, about 1.5 wt. % or more, about 2.5 wt. % or more, or about 2.7 wt. % or more up to about 6.3 wt. %, based on the weight of the pet food composition. In further embodiments, lignin is present in an amount of about 1.25 wt. % to 6 wt. %, about 1.25 wt. % to about 5 wt. %, about 1.25 wt. % to about 4 wt. %, or about 1.25 wt. % to about 3 wt. %, based on the weight of the pet food composition. In certain embodiments, lignin is present in an amount of about 1.25 wt. %, based on the weight of the pet food composition.

The pet food composition may comprise one or more botanicals. The botanicals may be present at various amounts or concentrations. As used here, the term “botanicals” refer to food or dietary supplements extracted or derived from a plant or herbal source that can provide health benefits and/or treat/prevent diseases. Some exemplary botanicals may include components such as, but not limited to, cranberry, pomace, ginger root, curcumin, green tea, pomegranate, chamomile, rosemary, aloe, nettle, Centella asiatica, Ginkgo biloba, Betula, witch hazel, grape skin extract, grape seed extract, grapefruit extract, grapefruit seed extract, bilberry extract, blueberry extract, soy isoflavones, black cohosh, St. John's Wort, echinacea, and chamomile. The botanicals may be extracted or derived from sources such as but not limited to acai, aloe vera, Asian ginseng, astragalus, bilberry, bitter orange, black cohosh, butterbury, cat's claw, chamomile, chasteberry, cinnamon, cranberry, candelion, echinacea, ephedra, European elder, European mistletoe, evening primrose oil, fenugreek, feverfew, flaxseed and flaxseed oil, garlic, ginger, ginkgo, goldenseal, grape seed extract, green tea, hawthorn, hoodia, horse chestnut, kava, lavender, licorice root, milk thistle, noni, passionflower, peppermint oil, red clover, sage, saw palmetto soy, St. John's Wort, tea tree oil, thunder god vine, turmeric, valerian, and yohimbe, and other sources listed in nlm.nih.gov/medlineplus/druginfo/herb_All.html. In certain preferred embodiments, the pet food composition comprises cranberry pomace, ginger root, pomegranate and green tea. In other embodiments, the pet food composition comprises cranberry pomace, ginger root, pomegranate, green tea and beet pulp.

In one embodiment, the one or more botanicals may be present in an amount of at least about 1.2 wt. %, based on the weight of the pet food composition. For example, botanicals may be present in an amount of about 1.2 weight %, about 1.5 weight %, about 1.8 weight %, about 2.0 weight %, about 2.5 weight %, about 3.0 weight %, about 3.5 weight %, about 4.0 weight %, about 4.5 weight %, about 5.0 weight %, about 5.5 weight %, about 6.0 weight %, about 6.5 weight %, or a range therebetween, based on the total weight of the pet food composition. In another example, botanicals may be present in an amount of from about 1.2 wt. % to about 6.0 wt. %, about 1.2 wt. % to about 5.5 wt. %, about 1.2 wt. % to about 5.0 wt. %, about 1.2 wt. % to about 4.5 wt. %, or about 1.2 wt. % to about 4.0 wt. %, based on the weight of the pet food composition. In further embodiments, botanicals are present in an amount of about 1.2 wt. % or more, about 1.5 wt. % or more, about 2.5 wt. % or more, or about 2.7 wt. % or more, up to about 6.5 wt. %, based on the weight of the pet food composition. In further embodiments, botanicals are present in an amount of about 1.2 wt. % to 6 wt. %, about 1.2 wt. % to about 5 wt. %, about 1.2 wt. % to about 4 wt. %, or about 1.2 wt. % to about 3 wt. %, based on the weight of the pet food composition. In certain embodiments, botanicals are present in an amount of about 1.2 wt. %, based on the weight of the pet food composition.

The one or more botanicals may be selected from erba Andrographis (Herba Andrographis), large Bulbus allii, Radix Et Caulis Acanthopanacis Senticosi (Siberian ginseng), the guaiacol component (derives from Oleum Cinnamomi (Cortex Cinnamomi), Flos caryophylli (clove tree, Flos caryophylli, fourth perfume (or spice)), or Cortex Cinnamomi (Cortex Cinnamomi, Cinnamomum zeylanicum Bl., Cortex Cinnamomi Bejolghotae, Radix Cinnamomi porrecti, bavin osmanthus, cloudy fragrant)), Borage seed oil (borage), Salvia japonica Thunb. (medicine Salvia japonica Thunb., Lavandula angustifolia, the leaf Salvia japonica Thunb., Salvia hispanolum), astragalus (Radix Astragali), Eupatorium perfoliatum (Eupatorium perfoliatum), Chamomile (Flos matricariae chamomillae, Chamaemelum nobile (L.) A L L), Cordyceps, Echinacea (Echinacea angustifolia, Echinacea, Echinacea), Ramulus sambuci Williamsii (Sambucus nigra L.), euphorbia, Radix ginseng (Radix panacis Quinquefolii, the Asia Radix ginseng, China's Radix ginseng, Radix ginseng, Radix ginseng: the Panax comprises Chinese Radix ginseng and Radix panacis Quinquefolii), white hair (canada yellow-root), Herba Chelidonii (mountain Rhizoma Coptidis), Wasabia japonica (Euterma Wasabi) (Radix cochleariae officinalis, the West Wasabia japonica (Euterma Wasabi)), Fructus actinidiae chinensis (green fruit Fructus actinidiae chinensis, Fructus actinidiae chinensis), dance mushroom (Grifola frondosa), Herba Visci (Visvum album L.), Herba Erodii (Flos pelargonii), Mentha arvensis L. syn.M.haplocalyxBrig/Oleum menthae (Mentha arvensis L. syn.M.haplocalyxBrig), propolis, red elm (red elm, rough branch elm), peppery (the Radix rumicis acetosae of folic acid, sheep's sorrel), Herba thymi vulgaris/Thymi Serpylli Herba extract (Herba thymi vulgaris), indigo grass (match indigo flower), quercetin (flavonol), and a combination of two or more thereof. Additionally or alternatively, the pet food composition may comprise one or more botanicals selected from Rhizoma Zingiberis Recens section (Zingiberaceae), Radix Glycyrrhizae, Althaea rosea (L.) Cavan. (Althaea officinalis L., Radix althaeae roseae), Chamomile (Flos matricariae chamomillae, Chamaemelum nobile (L.) A L L), fennel oil, Foeniculum vulgare (Fructus Foeniculi), caraway oil, Fructus cari carvi seed (caraway seed, Fructus cari carvi, caraway oil), Fructus Citri Limoniae face cream (Herba Melis sae officinalis leaf, Herba melis sae axillaris), Ou Xiazhi Rhizoma et Radix valerianae (Murrubii herba), Semen Lini α-linoleic acid (Semen Lini), and a combination of two or more thereof.

The compositions of the present invention may optionally comprise additional ingredients suitable for use in pet food compositions. Examples of such ingredients include, but are not limited to, protein, fat, carbohydrates, dietary fibers, amino acids, minerals, trace elements, vitamins, additives.

The pet food composition may comprise protein and/or a digestible crude protein. The term “protein” means a polypeptide, or a peptide, or a polymer of amino acids. The term encompasses naturally occurring and non-naturally occurring (synthetic) polymers and polymers in which artificial chemical mimetics are substituted for one or more amino acids. The term also encompasses fragments, variants, and homologs that have the same or substantially the same properties and perform the same or substantially the same function as the original sequence. The term encompasses polymers of any length, including polymers containing from about 2 to 1000, from 4 to 800, from 6 to 600, and from 8 to 400 amino acids. The term includes amino acid polymers that are synthesized and that are isolated and purified from natural sources. Under some embodiments, the terms “polypeptide”, “peptide” or “protein” are used interchangeably.

The protein and/or digestible crude protein of the composition may be present at various amounts or concentrations. In one embodiment, the protein may be present in an amount of from about 20 wt. % to about 45 wt. %, based on the weight of the pet food composition. For example, the protein may be present in an amount of about 20 weight %, about 25 weight %, about 30 weight %, about 35 weight %, about 40 weight %, or about 45 weight, or a range thereof, based on the total weight of the pet food composition. In another example, the protein may be present in an amount of from about 25 wt. % to about 40 wt. %, from about 30 wt. % to about 40 wt. %, or about 30 wt. % to about 35 wt. %, based on the weight of the pet food composition. In certain embodiments, the protein is present in an amount of about 20 wt. % to about 35 wt. %, about 25 wt. % to about 35 wt. %, or about 28 wt. % to about 35 wt. %, based on the weight of the pet food composition.

Digestible crude protein” is the portion of protein that is available or can be converted into free nitrogen (amino acids) after digesting with gastric enzymes. In vitro measurement of digestible crude protein may be accomplished by using gastric enzymes such as pepsin and digesting a sample and measuring the free amino acid after digestion. In vivo measurement of digestible crude protein may be accomplished by measuring the protein levels in a feed/food sample and feeding the sample to an animal and measuring the amount of nitrogen collected in the animal's feces.

A portion of the protein in the pet food composition may be digestible protein. For example, the composition may include an amount of protein, where about 40 weight % or more, about 50 weight % or more, about 60 weight % or more, about 70 weight % or more, about 80 weight % or more, about 90 weight % or more, about 95 weight % or more, about 98 weight % or more, or about 99 weight % or more of the protein is digestible protein. In some embodiments, e.g., when the composition desirable promotes weight loss, the portion of protein that is digestible protein is about 60 weight % or less, about 50 weight % or less, about 40 weight % or less, about 30 weight % or less, about 20 weight % or less, or about 10 weight % or less, based on the total amount of protein in the composition. In further embodiment, the amount of protein that is digestible protein is about 10 to about 99 weight %, about 10 to about 95 weight %, about 10 to about 90 weight %, about 10 to about 70 weight %, about 10 to about 50 weight %, about 10 to about 30 weight %; about 30 to about 99 weight %, about 30 to about 95 weight %, about 30 to about 90 weight %, about 30 to about 70 weight %, about 30 to about 50 weight %; about 50 to about 99 weight %, about 50 to about 95 weight %, about 50 to about 90 weight %, about 50 to about 70 weight %; or about 70 to about 99 weight %, about 70 to about 95 weight %, about 70 to about 90 weight %, including ranges and subranges therein, based on the total amount of protein in the composition.

Amino acids, including essential amino acids, can be added to the compositions of the present disclosure as free amino acids, or supplied by any number of sources, e.g., crude protein, to the compositions of the present disclosure. Essential amino acids are amino acids that cannot be synthesized de novo, or in sufficient quantities by an organism and thus must be supplied in the diet. Essential amino acids vary from species to species, depending upon the organism's metabolism. For example, it is generally understood that the essential amino acids for dogs and cats (and humans) are phenylalanine, leucine, methionine, lysine, isoleucine, valine, threonine, tryptophan, histidine and arginine. In addition, taurine, while technically not an amino acid but a derivative of cysteine, is an essential nutrient for cats.

The total dietary fiber of the composition may be present at various amounts or concentrations. In one embodiment, total dietary fiber may be present in an amount of less than wt. %, based on the weight of the pet food composition. In certain embodiments, the total dietary fiber is present in an amount of about 10 wt. % to about 20 wt. %, based on the weight of the pet food composition. For example, total dietary fiber may be present in an amount of about weight %, about 10.5 weight %, about 11.0 weight %, about 11.5 weight %, about 12.0 weight %, about 12.5 weight %, about 13.0 weight %, about 13.5 weight %, about 14.0 weight %, about 14.5 weight %, about 15.0 weight %, about 15.5 weight %, about 16.0 weight %, about 16.5 weight %, about 17.0 weight %, about 17.5 weight %, about 18.0 weight %, about 18.5 weight %, about 19.0 weight %, about 19.5 weight %, about 20.0 weight %, or a range therebetween, based on the total weight of the pet food composition. In another example, total dietary fiber may be present in an amount of from about 10 wt. % to about 18 wt. %, about 12 wt. % to about 18 wt. %, or about 15 wt. % to about 18 wt. %, based on the weight of the pet food composition. In further embodiments, total dietary fiber is present in an amount of about 15 wt. % to about 20 wt. %, about 16 wt. % to about 19 wt. %, or about 16 wt. % to about 18 wt. %, based on the weight of the pet food composition.

Carbohydrates can be supplied by any of a variety of sources known by those skilled in the art, including oat fiber, cellulose, peanut hulls, beet pulp, parboiled rice, corn starch, corn gluten meal, and any combination of those sources. Grains supplying carbohydrates can include, but are not limited to, wheat, corn, barley, and rice. Carbohydrates content of foods can be determined by any number of methods known by those of skill in the art. Generally, carbohydrate percentage can be calculated as nitrogen free extract (“NFE”), which can be calculated as follows: NFE=100%-moisture %-protein %-fat %-ash %-crude fiber %. The amount of carbohydrate, e.g., calculated as NFE, present in the pet food composition may be about 10 to about 90 weight %, about 10 to about weight %, about 10 to about 50 weight %, about 10 to about 40 weight %, about 10 to about 30 weight %, about 10 to about 20 wt. %; about 20 to about 90 weight %, about 20 to about 70 weight %, about 20 to about 50 weight %, about 20 to about 40 weight %; about 30 to about 90 weight %, about 30 to about 70 weight %, about 30 to about 50 weight %, about 30 to about 40 weight %; about 50 to about 90 weight %, about 50 to about 70 weight %; or about 70 to about 90 weight %, based on the total weight of the composition on a dry matter basis.

Dietary fiber refers to components of a plant which are resistant to digestion by an animal's digestive enzymes. Dietary fiber includes soluble and insoluble fibers. Soluble fibers are resistant to digestion and absorption in the small intestine and undergo complete or partial fermentation in the large intestine, e.g., beet pulp, guar gum, chicory root, psyllium, pectin, blueberry, cranberry, squash, apples, oats, beans, citrus, barley, fructooligosaccharides (FOS), or peas. Insoluble fibers can be supplied by any of a variety of sources, including, for example, cellulose, whole wheat products, wheat oat, corn bran, flax seed, grapes, celery, green beans, cauliflower, potato skins, fruit skins, vegetable skins, peanut hulls, rye berries, sweet potato, and soy fiber. Crude fiber includes indigestible components contained in cell walls and cell contents of plants such as grains, for example, hulls of grains such as rice, corn, and beans. Typical fiber amounts in compositions of the present disclosure can be from about 0 to 20 wt. %, or about 10 wt. % to about 20 wt. %, based on the total weight of the pet food composition. In certain embodiments, the total dietary fiber is present in an amount of about 10 wt. % to about 20 wt. %, based on the total weight of the pet food composition. For example, total dietary fiber may be present in an amount of about 10.0 weight %, about 10.5 weight %, about 11.0 weight %, about 11.5 weight %, about 12.0 weight %, about 12.5 weight %, about 13.0 weight %, about 13.5 weight %, about 14.0 weight %, about 14.5 weight %, about 15.0 weight %, about 15.5 weight %, about 16.0 weight %, about 16.5 weight %, about 17.0 weight %, about 17.5 weight %, about 18.0 weight %, about 18.5 weight %, about 19.0 weight %, about 19.5 weight %, or about 20.0 weight %. In another example, total dietary fiber may be present in an amount of from about 10% to about 18%, about 12% to about 18%, or about 15% to about 18%, based on the total weight of the pet food composition. In further embodiments, total dietary fiber is present in an amount of about 15% to about 20 wt. %, about 16% wt. to about 19 wt. %, or about 16 wt. % to about 18 wt. %, based on the total weight of the pet food composition on a dry matter basis.

In one embodiment, the pet food composition comprises one or more botanicals and beet pulp. In some embodiments, the botanicals are present in an amount of about 1.2 wt. % or more, about 1.5 wt. % or more, about 2.5 wt. % or more, or about 2.7 wt. % or more, up to about 6.5 wt. %, based on the weight of the pet food composition and the beet pulp is present in an amount of about 0.8 or more, about 1.0 wt. % or more, or about 1.3 wt. % or more, up to 5 wt. %, based on the weight of the pet food composition. In certain embodiments, the botanicals are present in an amount of about 1.2 wt. %, based on the weight of the pet food composition, and beet pulp is present in an amount of about 1.0 wt. %, based on the weight of the pet food composition.

The compositions of the present invention may optionally comprise fat. The term “fat” generally refers to a lipid or mixture of lipids that may generally be a solid or a liquid at ordinary room temperatures (e.g., 25° C.) and pressures (e.g., 1 atm). In some instances, the fat may be a viscous liquid or an amorphous solid at standard room temperature and pressure. Fat can be supplied by any of a variety of sources known by those skilled in the art, including meat, meat by-products, canola oil, fish oil, and plants. Plant fat sources include wheat, flaxseed, rye, barley, rice, sorghum, corn, oats, millet, wheat germ, corn germ, soybeans, peanuts, and cottonseed, as well as oils derived from these and other plant fat sources. The compositions of the present disclosure may contain at least about 9 wt. % (or from about 9 wt. % to about 25 wt. %, or from about 10 wt. % to about 20 wt. %, or from about 10 wt. % to about 15 wt. %) total fat, based on the total weight of the pet food composition. In some cases, the fat in the compositions is crude fat. Crude fat may be included into the compositions in amounts of from about 10 to about 20 weight %, about 10 to about 18 weight %, about 10 to about 16 weight %; about 12 to about 20 weight %, about 12 to about 18 weight %, or about 12 to about 16 weight %, based on the total weight of the composition on a dry matter basis. In some cases, it may be preferable that about 50 weight % or more, about weight % or more, about 70 weight % or more, about 80 weight % or more, or about 90 weight % or more of the total fat is obtained from an animal source. Alternatively, about 50 weight % or more, about 60 weight % or more, about 70 weight % or more, about 80 weight % or more, or about 90 weight % or more of the total fat may be obtained from a plant source.

The compositions of the present disclosure can also contain one or more minerals and/or trace elements, e.g., calcium, phosphorus, sodium, potassium, magnesium, manganese, copper, zinc, chromium, molybdenum, selenium, or iron salts having counter ions such as, for example chloride, iodide, fluoride, sulfide or oxide, in amounts required to avoid deficiency and maintain health. These amounts are known by those of skill in the art, for example, as provided in the Official Publication of the Associate of American Feed Control Officials, Inc. (“AAFCO”) 2020, or the National Research Council, 2006, Nutrient requirements of dogs and cats, National Academies Press. Typical mineral amounts are about 0.1% to about 4% or about 1% to about 2%.

The compositions of the present invention can also include vitamins in amounts required to avoid deficiency and maintain health. These amounts and methods of measurement are known by those skilled in the art. For example, the Official Publication of the Associate of American Feed Control Officials, Inc. (“AAFCO”), Nutrient Requirements of Dogs and Cats, 2020, provides recommended amounts of such ingredients for dogs and cats. As contemplated herein, vitamins can include, but are not limited to, vitamin A, vitamin B₁, vitamin B₂, vitamin B₆, vitamin B₁₂, vitamin C, vitamin D, vitamin E, vitamin H (biotin), vitamin K, folic acid, choline, inositol, niacin, and pantothenic acid. Typical vitamin amounts in the composition of the invention are about from wt. % to about 3 wt. % or about 1 wt. % to about 2 wt. %, based on the total weight of the pet food composition.

The compositions of the present disclosure can additionally comprise other additives such as palatability enhancers and stabilizers in amounts and combinations familiar to one of skill in the art. Stabilizing substances include, for example, substances that tend to increase the shelf life of the composition. Other examples of other such additives potentially suitable for inclusion in the compositions of the invention include, for example, preservatives, colorants, antioxidants, flavorants, synergists and sequestrants, packaging gases, stabilizers, emulsifiers, thickeners, gelling agents, and humectants. Examples of emulsifiers and/or thickening agents include, for example, gelatin, cellulose ethers, starch, starch esters, starch ethers, and modified starches. The concentration of such additives in the composition typically can be up to about 5% by weight, based on the total weight of the pet food composition. In some embodiments, the concentration of such additives (particularly where such additives are primarily nutritional balancing agents, such as vitamins and minerals) is from about 0% to about 2.0% by weight, based on the total weight of the pet food compositions. In some embodiments, the concentration of such additives (again, particularly where such additives are primarily nutritional balancing agents) is from about 0% to about 1.0% by weight, based on the total weight of the pet food compositions.

Foods of any consistency or moisture content are contemplated, e.g., the compositions of the present invention can be, for example, a dry, moist or semi-moist animal food composition. In some embodiments, the moisture content is from about 3 wt. % to about 90 wt. % of the total weight of the composition. “Semi-moist” refers to a food composition containing from about 25 wt. % to about 35 wt. % moisture, based on the total weight of the pet food composition. “Moist” food refers to a food composition that has a moisture content of about 60 wt. % to 90 wt. % or greater, based on the total weight of the pet food composition. “Dry” food refers to a food composition with about 3 wt. % to about 12 wt. %, based on the total weight of the pet food composition, of moisture content and is often manufactured in the form of small bits or kibbles.

The moisture of the composition may be present at various amounts or concentrations. The moisture may be present at various amounts or concentrations. In one embodiment, the moisture may be present in an amount of from about 5 wt. % to about 15 wt. %, based on the weight of the pet food composition. For example, the moisture may be present in an amount of about 5.0 weight %, about 5.5 weight %, about 6.0 weight %, about 6.5 weight %, about 7.0 weight %, about 7.5 weight %, about 8.0 weight %, about 8.5 weight %, about 9.0 weight %, about 9.5 weight %, about 10.0 weight %, about 10.5 weight %, about 11.0 weight %, about 11.5 weight %, about 12.0 weight %, about 12.5 weight %, about 13.0 weight %, about 13.5 weight %, about 14.0 weight %, about 14.5 weight %, about 15.0 weight %, or a range thereof, based on the total weight of the pet food composition. In another example, the moisture may be present in an amount of from about 8 wt. % to about 13 wt. %, about 9 wt. % to about 13 wt. %, about 9 wt. % to about 11 wt. %, or about 9 wt. % to about 13 wt. %, based on the weight of the pet food composition. In certain embodiments, the moisture is present in an amount of about 10 wt. % to about 12 wt. %, about 10.5 wt. % to about 12 wt. %, or about 10.5 wt. % to about 11.5 wt. %, based on the weight of the pet food composition.

In certain aspects, the present application further discloses a method of making any of the compositions of the present disclosure. In preparing a composition of the present invention in wet or canned form, any ingredient (e.g., soluble fiber and desired ratio of linolenic acid):(total 18 carbon polyunsaturated fatty acids) generally can, for example, be incorporated into the composition during the processing of the formulation, such as during and/or after mixing of other components of the composition. Distribution of these components into the composition can be accomplished by conventional means. In some embodiments, ground animal and poultry proteinaceous tissues are mixed with the other ingredients, including fish oils, cereal grains, other nutritionally balancing ingredients, special-purpose additives (e.g., vitamin and mineral mixtures, inorganic salts, cellulose and beet pulp, bulking agents, and the like); and water that is sufficient for processing is also added. These ingredients can be mixed in a vessel suitable for heating while blending the components. Heating of the mixture can be effected using any suitable manner, such as, for example, by direct steam injection or by using a vessel fitted with a heat exchanger. Following the addition of the last ingredient, the mixture can be heated to a temperature range of from about 50° F. (10° C.) to about 212° F. (100° C.). In some instances, the mixture can be heated to a temperature range of from about 70° F. (21° C.) to about 140° F. (60° C.). Temperatures outside these ranges are generally acceptable but may be commercially impractical without use of other processing aids. When heated to the appropriate temperature, the material will typically be in the form of a thick liquid. The thick liquid can be filled into cans. When filled into cans, a lid is applied, and the container is hermetically sealed. The sealed can is then placed into conventional equipment designed to sterilize the contents. This is usually accomplished by heating to temperatures of greater than about 230° F. (110° C.) for an appropriate time, which is dependent on, for example, the temperature used and the composition.

Pet food compositions can alternatively be prepared in a dry form using conventional processes. Typically, dry ingredients, including, for example, animal protein, plant protein, grains, etc., are ground and mixed together. Moist or liquid ingredients, including fats, oils, animal protein, water, etc., are then added to and mixed with the dry mix. The mixture is then processed into kibbles or similar dry pieces. Kibble is often formed using an extrusion process in which the mixture of dry and wet ingredients is subjected to mechanical work at a high pressure and temperature, then forced through small openings and cut off into kibble by a rotating knife. The wet kibble is then dried and optionally coated with one or more topical coatings which may include, for example, flavors, fats, oils, powders, and the like. Kibble also can be made from the dough using a baking process, rather than extrusion, wherein the dough is placed into a mold before dry-heat processing.

In another aspect, the present disclosure provides for a method for decreasing the weight in a companion animal, comprising feeding an effective amount of a composition as described herein. The companion animal may be a dog or cat.

In another aspect, the present disclosure provides for a method for decreasing the fat in a companion animal, comprising feeding an effective amount of a composition as described herein. The companion animal may be a dog or cat.

In certain embodiments, the lean mass of the companion animal is maintained while the fat is decreased. As such, in certain embodiments the present disclosure provides for a method for increasing the lean to fat ratio in a companion animal, comprising feeding an effective amount of a composition as described herein. The companion animal may be a dog or cat.

### Example

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

Eighteen healthy cats, considered to have no evidence of a chronic systemic disease, were fed either a control composition or test composition. The studies comprised two phases. The first phase included feeding the cat an amount of food to maintain body weight from a baseline time point to a post-intervention weight maintenance time point. In the second phase, the cats were fed a pet food composition in an amount to lose approximately 0.75-1% of total body weight from the post-intervention weight maintenance time point to a post-intervention weight loss time point. To accomplish this, body weight was measured during the pre-feed period and food offering was calculated as a percent of the observed maintenance energy requirement (that number of calories required for weight maintenance) in order to optimize weight loss through offering the amount of food that would result in the intended rate of loss. This was often about 90% of the calories required for maintenance. Specifically, the resting energy requirement (RER) was determined by the formula (70*kg ideal body weight^{0.75}). Subsequently, the modulating activity factor was scaled from the original value that supported the obese condition (Activity Factor 1.2-1.8) to a value ˜90% lower. Each week the subject was reweighed and the activity factor scaled to increase (or decrease) the rate of weight loss if the rate was less (or more) than the target rate of 0.75-1% body weight per week. Details of the pet food compositions are summarized in Table 1 and Table 2.

As used herein, “Control” refers to a comparative example and “Test” refers to an exemplary composition of the present invention.

TABLE 1
Pet food compositions (wt. %).

| | Control | Test |
|---|---|---|
| Chicken | 25.8 | 20.7 |
| Pea protein | 11.4 | 14.8 |
| Yellow split peas | 11.7 | 12.6 |
| Potato starch | 11.1 | 11.1 |
| Oat Fiber | 9.0 | 6.0 |
| Oat enriched beta glucan (1-3, 1-4, beta | 0.0 | 3.0 |
| glucan source) | | |
| Yeast beta glucan enriched (1-3, 1-6 beta | 0.0 | 0.2 |
| glucan source) | | |
| Egg | 7.6 | 5.5 |
| Tomato Pomace | 5.0 | 5.0 |
| Cellulose | 4.0 | 0.0 |
| Flax Seed | 3.0 | 2.0 |
| Chicken liver | 2.5 | 2.5 |
| Coconut Oil (C8 and C10 source) | 1.8 | 1.8 |
| Fish Oil (EPA and DHA source) | 0.0 | 1.2 |
| Pecan fiber (lignin source) | 0.0 | 3.0 |
| Fruits and vegetables mix | 1.1 | 1.1 |
| Ginger root, Pomegranate (botanical source) | 0.0 | 1.2 |
| Lactic acid | 1.2 | 1.2 |
| Minors (Palatant, Vitamins, Minerals, Amino | 4-5 (e.g. 4.8) | 5-6 (e.g. 5.6) |
| Acids) | | |
| Betaine | 0.0 | 0.5 |
| Beet pulp | 0.0 | 1.0 |

TABLE 2
Food composition Analyte Quantification.

| | Control | Test |
|---|---|---|
| Caprylic acid (C8) | 0.10 | 0.10 |
| Capric acid (C10) | 0.08 | 0.08 |
| EPA (C20) | 0.01 | 0.18 |
| DHA (C22) | 0.04 | 0.14 |
| Betaine | 0.01 | 5.00 |
| Total dietary fiber | 18.90 | 16.80 |
| Soluble fiber | 1.00 | 0.70 |

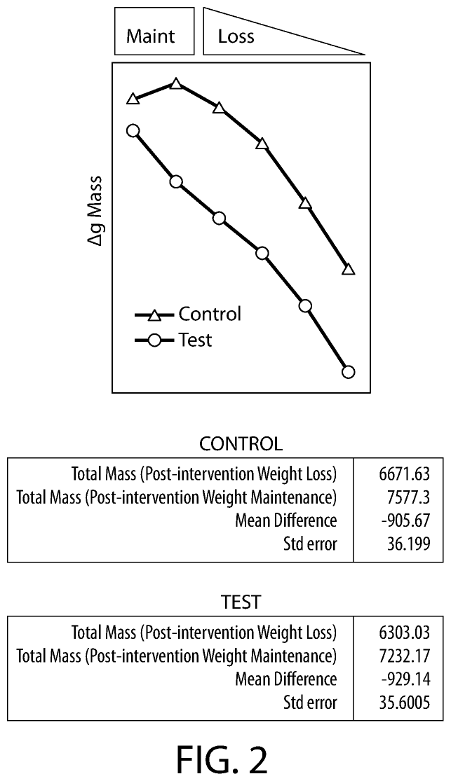

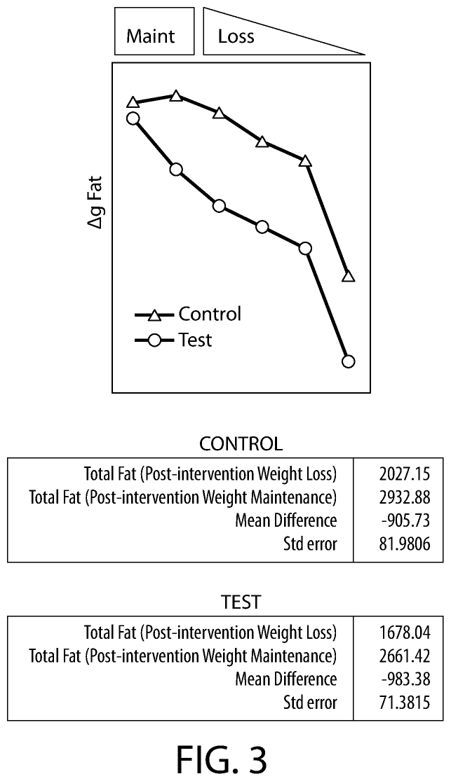

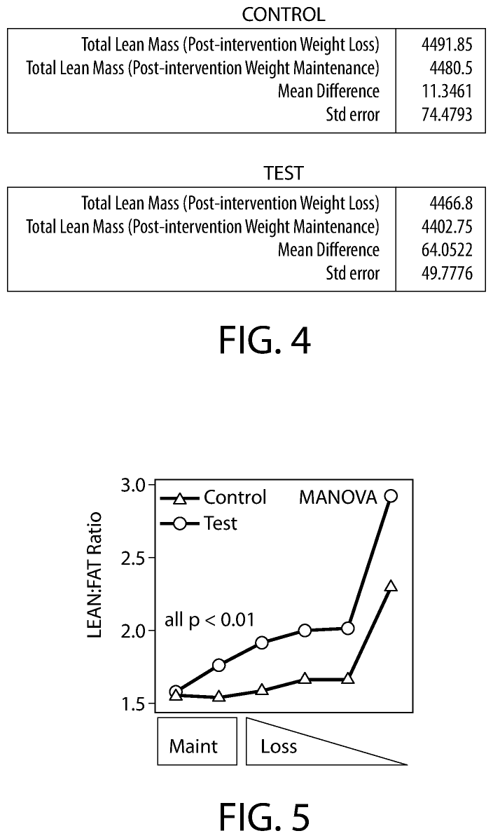

FIG. 2 shows the effects on mass of felines fed the test diet. Weight loss is shown to be greater for the test diet when compared to the control diet. FIG. 3 shows the effects on animal fat of felines fed the test diet. Fat loss is shown to be greater for the test diet when compared to the control diet. FIG. 4 shows the effects on lean mass of felines fed the test diet. Lean mass is shown to increase more for the test diet when compared to the control diet. FIG. 5 shows the effects on the lean to fat ratio of felines fed the test diet. The lean to fat ratio is greater for animals consuming the test diet when compared to the control diet. These surprising and unexpected results show that animals fed with the test diet had enhanced weight loss, fat loss, and yet also retained lean mass. Such results yield improved animal lean to fat ratios.

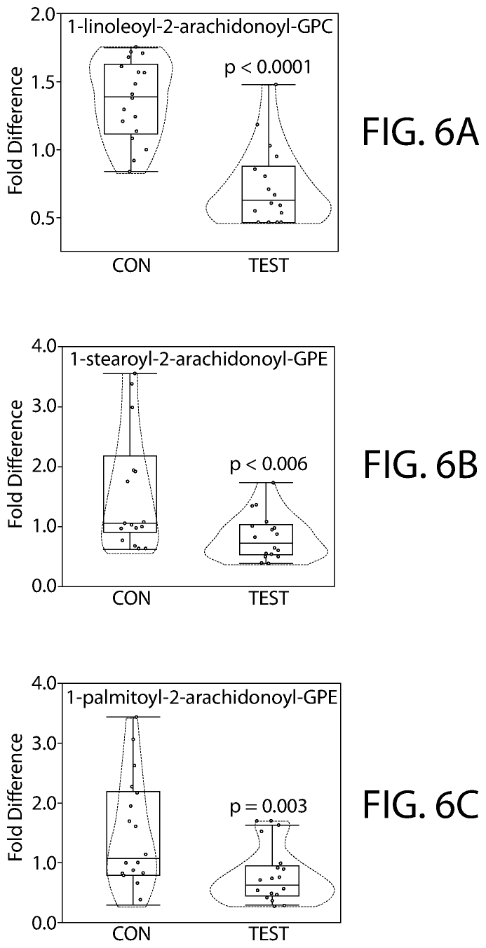

FIG. 6A shows effects on 1-linoleoyl-2-arachidonoyl-GPC of felines fed the test diet. FIG. 6B shows effects on 1-stearoyl-2-arachidonoyl-GPE of felines fed the test diet. FIG. 6C shows effects on 1-palmitoyl-2-arachidonoyl-GPE of felines fed the test diet. These figures show that the test food decreased arachidonate precursors of inflammatory prostaglandins and leukotrienes. Significantly, these decreased results occurred in conjunction with achieved animal weight loss. Interestingly, the anti-inflammatory endocannabinoid 2-AG was observed to be higher in some time points within the test food composition, when compared to the control food composition (data not shown).

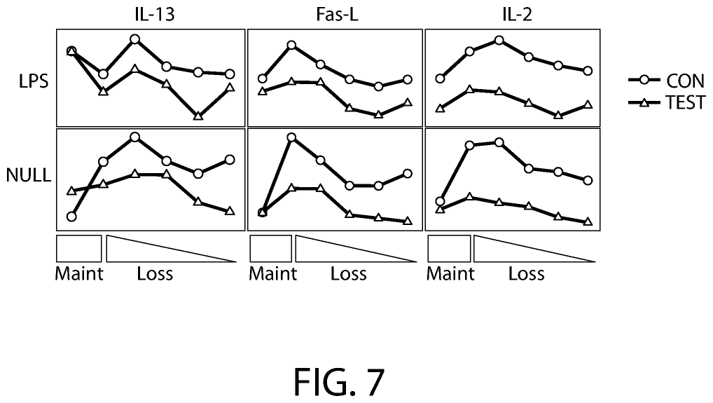

FIG. 7 shows that the test food composition decreases various inflammatory cytokine levels, when compared to the control food composition.

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

1. A pet food composition comprising:
1-3, 1-6 beta glucan and 1-3, 1-4 beta glucan present in a total amount equal to about 0.8% or more, based on the weight of the pet food composition, wherein the 1-3, 1-4 beta glucan is about 83% or more, by weight, of the two beta glucans;
two or more medium chain saturated fatty acids;
two or more omega-3 polyunsaturated fatty acids present in a total amount of about 0.5% or more, based on the weight of the pet food composition, wherein the two or more medium chain saturated fatty acids are at least 36%, by weight, of these fatty acids; and
betaine present in an amount of about 0.5% or more, based on the weight of the pet food composition.

2. The pet food composition according to claim 1, wherein 1-3, 1-6 beta glucan and 1-3, 1-4 beta glucan are present in an amount of from about 0.8% to about 5%, based on the weight of the pet food composition.

3. (canceled)

4. The pet food composition according to claim 1, wherein the 1-3, 1-4 beta glucan is present in an amount from 83% to 95%, by weight, of the two beta glucans.

5. (canceled)

6. The pet food composition according to claim 1, wherein the two or more medium chain saturated fatty acids comprise a C8 fatty acid and a C10 fatty acid; or wherein the two or more omega 3 polyunsaturated fatty acids comprise eicosapentaenoic acid (EPA) and docosahexaenoic acid (DHA).

7. (canceled)

8. The pet food composition according to claim 1, wherein the two or more medium chain saturated fatty acids and the two or more omega 3 polyunsaturated fatty acids are present in a total amount of from about 0.5% to about 7%, based on the weight of the pet food composition.

9. (canceled)

10. The pet food composition according to claim 6, wherein the C8 and C10 comprise from about 36% to about 60%, by weight, of the total amount of the two or more medium chain saturated fatty acids and the two or more omega 3 polyunsaturated fatty acids.

11. (canceled)

12. (canceled)

13. The pet food composition according to claim 1, wherein the betaine is present in an amount of about 0.5% to about 2%, based on the weight of the pet food composition.

14. (canceled)

15. The pet food composition according to claim 1, further comprising lignin present in an amount of at least 1.25%, based on the weight of the pet food composition.

16. (canceled)

17. (canceled)

18. The pet food composition according to claim 1, further comprising botanicals.

19. The pet food composition according to claim 18, wherein the botanicals comprise cranberry pomace, ginger root, pomegranate and green tea.

20. The pet food composition according to claim 19, wherein the cranberry pomace, ginger root, pomegranate and green tea are present in a total amount of about 1.2% or more, based on the weight of the pet food composition.

21. The pet food composition according to claim 1, further comprising beet pulp.

22. The pet food composition according to claim 21, wherein the beet pulp is present in an amount of about 1% or more, based on the weight of the pet food composition.

23. (canceled)

24. A method for increasing the lean to fat ratio in a companion animal, comprising feeding an effective amount of the pet food composition of claim 1.

25-46. (canceled)

47. A pet food composition comprising:
one or more beta glucan present in a total amount of about 0.8% or more, based on the weight of the pet food composition;
one or more medium chain saturated fatty acids;
one or more omega-3 polyunsaturated fatty acids present in a total amount of about 0.5% or more, based on the weight of the pet food composition; and
betaine present in an amount of about 0.5% or more, based on the weight of the pet food composition.
